(19) United States
(12) Patent Application Publication
(10) Pub. No.: US 2011/0208409 A1
(43) Pub. Date: Aug. 25, 2011

(54) FUEL BLEND SENSING SYSTEM

(76) Inventors: David Benjamin Snyder, Indianapolis, IN (US); Gregory Matthew Shaver, Lafayette, IN (US)

(21) Appl. No.: 13/056,732

(22) PCT Filed: Aug. 3, 2009
(86) PCT No.: PCT/US09/52613

§ 371 Date: May 2, 2011

Related U.S. Application Data
(60) Provisional application No. 61/085,608, filed on Aug. 1, 2008.

(51) Int. Cl. F02D 41/00; F02D 41/18; F02D 41/26; F02D 41/30; G01N 33/22

(52) U.S. Cl. 701/109; 123/703; 701/108; 123/698; 123/672; 123/674

(57) ABSTRACT

While the materials compatibility challenges have largely been met in “flex-fuel” vehicles, the engine and aftertreatment operation has not been optimized as function of fuel type (i.e. ethanol, biodiesel, etc.). The full-scale introduction of alternative fuels is most likely going to occur as blends with conventional fuels. This is seen to some extend with the limited introduction of E85 (85% ethanol, 15% gasoline) and B20 (20% biodiesel, 80% conventional diesel.). This further exacerbates the challenge of accommodating variable fuel properties, as there will be differences in combustion properties due to both the type of alternative fuel (i.e. pure biodiesel vs. pure diesel) and blend ratio (i.e. B20 vs. B80). Real-time estimation of the fuel blend is key to the optimized use of two-component fuels (e.g. diesel-biodiesel, gasoline-ethanol, etc.). The approach outlined here uses knowledge of the exhaust composition, fuel and air delivery rates to the engine to estimate the fuel blend. The strategy is illustrated with a production wideband O₂ in the engine's exhaust stream, coupled with the knowledge of the air-fuel ratio, to estimate the percentage of biodiesel in fuel being delivered to a 2007 Cummins turbo-diesel engine.

### CROSS REFERENCE TO RELATED APPLICATION

This application claims the benefit of priority to U.S. Provisional Patent Application Ser. No. 61/085,608, filed Aug. 1, 2009, entitled BIOFUEL BLEND SENSOR, incorporated herein by reference.

### FIELD OF THE INVENTION

The present inventions pertain to measurement, analysis, and control algorithms for internal combustion engines, and in particular to control algorithms for internal combustion engines using any two-component fuels in which the fuels have different stoichiometric mixture fractions.

### BACKGROUND OF THE INVENTION

While the materials compatibility challenges have largely been met in “flex-fuel” vehicles, the engine and aftertreatment operation has not been optimized as function of fuel type (i.e. ethanol, biodiesel, etc.). The full-scale introduction of alternative fuels is most likely going to occur as blends with conventional fuels. This is seen already to some extend with the limited introduction of E85 (85% ethanol, 15% gasoline) and B20 (20% biodiesel, 80% conventional diesel.). This further exacerbates the challenge of accommodating different fuel properties, as there will be differences in combustion properties due to both the type of alternative fuel (i.e. pure biodiesel vs. pure diesel) and blend ratio (i.e. B20 vs. B80)—see FIG. 1, for instance. Real-time estimation of the fuel blend is key to the optimized use of two-component fuels (e.g. diesel-biodiesel, gasoline-ethanol, etc.).

Therefore, what is needed is a generalizable strategy for determining the blend fraction of two-component fuels (e.g., biodiesel-diesel, ethanol-gasoline, etc.). This document presents novel and unobvious ways of accomplishing this.

### SUMMARY OF THE INVENTION

One aspect of the present invention pertains to an apparatus of an internal combustion engine including an air intake and a fuel source for combustion by the engine, the engine producing an exhaust flow from the combustion. Yet other embodiments include an oxygen sensor disposed in the exhaust flow for providing a signal representative of free oxygen content. Still other embodiments include a controller responsive to the signal.

Another aspect of the present invention pertains to a method of operating an internal combustion engine. Other embodiments include providing an internal combustion engine, and a mixed fuel. Further embodiments include operating the engine with the mixed fuel, and measuring the free oxygen content of the exhaust gas from the engine, and interpreting the ratio of the first fuel to the second fuel from the oxygen content.

Another aspect of the present invention pertains to a method of operating an internal combustion engine. The method further includes providing an internal combustion engine. Yet other embodiment include providing a first mixed fuel and a second mixed fuel, the first mixture ratio being different than the second mixture ratio. Another embodiment pertains to providing a general relationship of fuel mixture ratio to the free oxygen content of the engine exhaust gas and also to at least one of the engine airflow rate or the engine fuel flow rate. Yet other embodiments pertain to modifying the general relationship with data obtained by operating the engine.

It will be appreciated that the various apparatus and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is excessive and unnecessary.

### BRIEF DESCRIPTION OF THE DRAWINGS

FIG. 1 is a graphical representation of the average impact of biodiesel blends on emissions from pre-1998 heavy-duty on-highway engines in 2002 EPA study.

FIG. 2 is a graphical representation of the proposed two-input, one output approach for steady state biodiesel blend estimation.

FIG. 3 is a graphical representation according to one embodiment of the present invention of model predictions: O₂ vs. mixture fraction for conventional diesel (B0) and soy methyl ester biodiesel (B100).

FIG. 4 is a graphical representation according to one embodiment of the present invention of model predictions: O₂ vs. mixture fraction for soy methyl ester biodiesel blends B0, B20, B40, B60, B80, & B100.

FIG. 5 is a graphical representation according to one embodiment of the present invention of O₂ vs. mixture fraction using both the direct model and the simplified best fit model.

FIG. 6 is a graphical representation according to one embodiment of the present invention of contour plots of differences between direct estimator, Eqn. (8), and simplified best fit estimator, Eqn. (13)

FIG. 7 is a photographic representation of an engine used for steady-state experimental validation of a sensing system according to one embodiment of the present invention: a 6.7-liter 2007 Cummins ISB

FIG. 8 is a graphical representation according to one embodiment of the present invention of experimental data collection torque-speed points.

FIG. 9 is a graphical representation according to one embodiment of the present invention of experimental results: O₂ vs. mixture fraction for B0, B20, B50, and B100.

FIG. 10 is a schematic representation of a diesel engine according to one embodiment of the present invention.

FIG. 11 is a block diagram of a diesel engine system according to one embodiment of the present invention.

FIG. 12 is a graphical representation according to one embodiment of the present invention of the properties of different fuels.

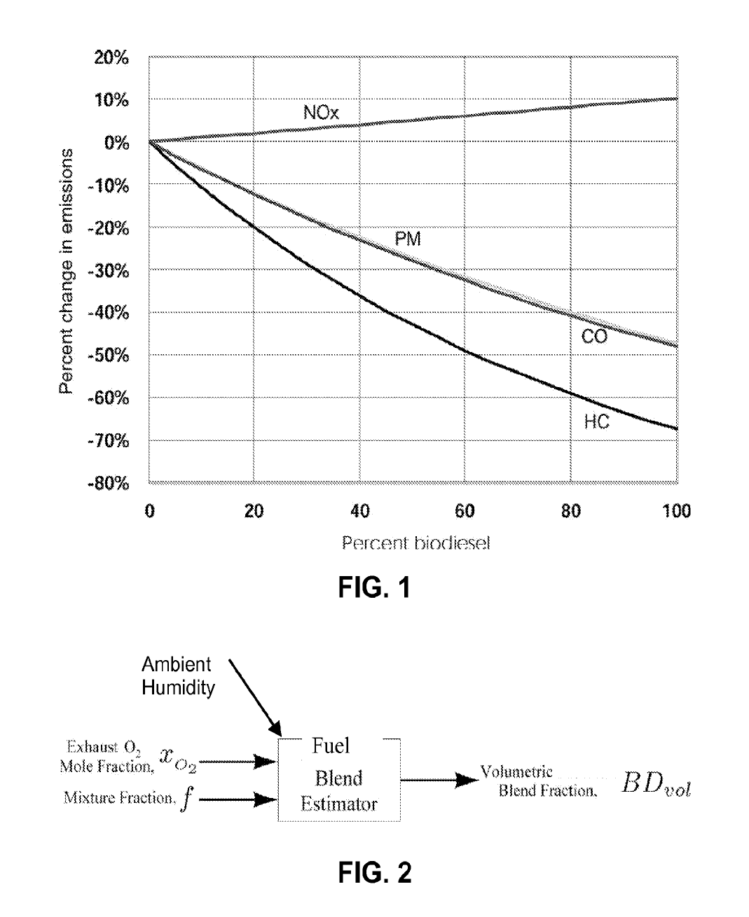

### DESCRIPTION OF THE PREFERRED EMBODIMENT

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. At least one embodiment of the present invention will be described and shown, and this application may show and/or describe other embodiments of the present invention. It is understood that any reference to “the invention” is a reference to an embodiment of a family of inventions, with no single embodiment including an apparatus, process, or composition that must be included in all embodiments, unless otherwise stated.

The use of an N-series prefix for an element number (NXX.XX) refers to an element that is the same as the non-prefixed element (XX.XX), except as shown and described thereafter. As an example, an element 1020.1 would be the same as element 20.1, except for those different features of element 1020.1 shown and described. Further, common elements and common features of related elements are drawn in the same manner in different figures, and/or use the same symbology in different figures. As such, it is not necessary to describe the features of 1020.1 and 20.1 that are the same, since these common features are apparent to a person of ordinary skill in the related field of technology. Although various specific quantities (spatial dimensions, temperatures, pressures, times, force, resistance, current, voltage, concentrations, wavelengths, frequencies, heat transfer coefficients, dimensionless parameters, etc.) may be stated herein, such specific quantities are presented as examples only. Further, with discussion pertaining to a specific composition of matter, that description is by example only, does not limit the applicability of other species of that composition, nor does it limit the applicability of other compositions unrelated to the cited composition.

Various embodiments of the present invention pertain to methods and apparatus that permit detection of the use of a blended fuel during operation of an internal combustion engine. In some embodiments, the mixed fuel being combusted in the engine is a blend of a first fuel containing little or no oxygen with a second hydrocarbon fuel that includes oxygen bonded within the fuel molecules. However, yet other embodiments of the present invention are not so constrained, and pertain to mixed fuels in which both, or neither of the, fuels contain bonded oxygen atoms.

Various embodiments of the present invention include means for detecting free oxygen molecules in the combustion by-products of the engine. For example, some embodiments include a wide-band O2 sensor, preferably located in the exhaust manifold of the engine. In yet other embodiments, the means for detecting oxygen can be located anywhere, but is in fluid communication with the exhaust flow of the engine. Further, means for detecting oxygen includes not just wide-band O2 sensors, but any transducer that produces a signal corresponding to the presence of molecular free oxygen.

Yet other embodiments of the present invention pertain to methods for relating the operating state of the engine to a fuel blend that is a mixture of fuels that have different stoichiometric mixture fractions. As one example, the different quantities used for establishing the state of the engine include measurements for one or more of the following: engine airflow, engine fuel flow, torque, speed, exhaust gas recirculation, fuel delivery parameters (such as pressure or timing parameters), intake or exhaust valve parameters (such as lift, duration, and other timing parameters), and the free oxygen content of the exhaust gas. These various state parameters can be used to estimate and quantify the blend of the two fuels.

In yet other embodiments there is a method for operating an internal combustion engine that includes measurement of free oxygen in the exhaust, and calculation of one or more of the following from the various state parameters: engine airflow and engine fuel flow. In some embodiments the calculated airflow and fuel flow (either as directly measured, inferred from other state parameters, or a combination of both) are used in a control algorithm that includes software coding that pertains to an engine combustion model. In some embodiments the higher-level definition of the software includes a simplified combustion model, or terms from a simplified combustion model. In yet other embodiments, the lower-level coding (such as object coding) includes data representative of teachings of the simplified combustion model (such as air-fuel ratio, oxygenated fuel fraction, non-oxygenated fuel fraction, numbers of particular atoms in a representative fuel molecule, oxygen mole fractions, and others shown herein).

Yet another embodiment of the present invention pertains to a method for calibrating or training a simplified combustion model. In one embodiment a simplified combustion model is prepared for an internal combustion engine. This simplified relationship includes one or more terms, such as a term that includes both a constant coefficient and also a calculated quantity (such as engine airflow, engine fuel flow, or free oxygen content of the exhaust). This relationship can be a generalized relationship that does not take into account specific features of a particular engine, and therefore lacks a desired accuracy in its predictive capability. One embodiment of the present invention includes preparing such a generalized relationship, and operating the internal combustion engine with a predetermined blend of two fuels having different stoichiometric mixture fractions. During operation of the engine with the blended fuel, various state measurements can be taken, and this measured data can be used to adjust the constant coefficients and improve the predictive accuracy of the simplified relationship. This improved simplified relationship is thereby made a specific relationship that pertains to specific characteristics of the tested engine.

Yet another embodiment of the present invention pertains to preparing an engine control software algorithm capable of modifying the desired operational characteristics of the engine based on a measurement of free oxygen content in the exhaust gas and a prediction of the manner in which the combusted fuel has been blended from two different fuels. In one embodiment, the software includes coding that corresponds to a generalized combustion model for an engine. In some embodiments, the generalized combustion model is simplified to include terms that are measurable quantities during operation of the engine. Two examples of simplified and generalized relationships are equations (A) and (B) shown herein. The relationship includes coefficients that multiply the calculated engine state parameters. In one embodiment, these coefficients are chosen based on data obtained from one or more engines of a particular family of engines (for example, a “family” of engines may be defined by a particular part number, a particular parts list or a name, including a trademarked name). These operationally established coefficients, along with the corresponding terms of the simplified relationship, are used in software that is programmed into the electronic control modules used in that family of engines.

One embodiment of the present invention pertains to a diesel engine combusting a mixture of conventional diesel fuel and biodiesel. Another embodiment pertains to a spark-ignited engine combusting a mixture of conventional gasoline fuel and ethanol. Yet other embodiments pertain to any internal combustion engine combusting a mixture of two component fuels in which the fuels have different stoichiometric mixture fractions. Preferably, the internal combustion engines operate with a lean mixture of air and blended fuels. Although what will be shown and described herein are analytical and test results using a diesel engine, the present invention is not so limited. Yet other embodiments contemplate usage of the methods and apparatus disclosed herein to reciprocating spark ignition engines, rotary spark ignition engines (such as a Wankel engine) and gas turbines.

The engine control system includes a wideband O₂ sensor that measures the free oxygen in the engine's exhaust stream. The engine controller executes an algorithm that estimates the amount of biodiesel (ethanol) fuel mixed with the conventional diesel (gasoline) fuel, and subsequently adapts other control algorithms, such as for engine fuel flow as a function of speed, torque, or other operating conditions for proper performance of the engine with mixed fuel. Many other engine control algorithms can include control schedules that can be improved with knowledge of the blend ratio of the fuel, including emission control schedules, engine starting algorithms, engine acceleration algorithms, and engine steady-state control schedules.

Yet another embodiment of the present invention pertains to a simplified algorithm for estimating the mixture ratio of a fuel that includes a first fuel with a second hydrocarbon fuel with a different stoichiometric mixture fraction. The algorithm includes an estimate of the fuel flow rate into the engine, an estimate of the airflow rate into the engine, and a measurement of the molecular oxygen in the exhaust gas. With knowledge of these three quantities, it is possible to estimate the mixture ratio of the two hydrocarbon fuels.

Yet another embodiment of the present invention pertains to a simplified algorithm for analyzing a fuel being burned by an internal combustion engine. The algorithm relates the ratio of biodiesel (or ethanol) fuel to conventional diesel (or gasoline) fuel as being proportional to the free oxygen content of the exhaust gas, proportional to the airflow into the cylinders of the engine, and proportional to the rate at which the mixed fuel is combusted. In some embodiments, the algorithm includes a term linearly proportional to the product of the oxygen content and the airflow rate divided by the fuel flow rate. In yet other embodiments the algorithm includes a second term linearly proportional to oxygen content and a third term linearly proportional to air fuel ratio.

The description herein references the engine controller for simplicity of description, which may be a standard type of Engine Control Module (ECM), including one or more types of memory or of a different configuration. The controller can be an electronic circuit comprised of one or more components, including digital circuitry, analog circuitry, or both. The controller may be a software and/or firmware programmable type; a hardwired, dedicated state machine; or a combination of these. In one embodiment, the controller is a programmable microcontroller solid-state integrated circuit that integrally includes one or more processing units and memory. Memory (if present) can be comprised of one or more components and can be of any volatile or nonvolatile type, including the solid state variety, the optical media variety, the units, and/or to provide for parallel or pipelined processing if desired. The controller functions in accordance with operating logic/algorithms defined by programming, hardware, or a combination of these. In one form, memory stores programming instructions executed by a processing unit of the controller to embody at least a portion of this operating logic. Alternatively or additionally, memory stores data that is manipulated by the operating logic of the controller. The controller can include signal conditioners, signal format converters (such as analog-to-digital and digital-to-analog converters), limiters, clamps, filters, and the like as needed to perform various control and regulation operations described in the present application. The controller receives various inputs and generates various outputs to perform various operations as described hereinafter in accordance with its operating logic.

Like biodiesel, ethanol is an oxygenated, domestically available alternative fuel, which has different combustion properties than the conventional fuel (gasoline for ethanol, diesel for biodiesel) for which it is an alternative. The high octane number of ethanol is what makes it an appropriate alternative to gasoline in spark-ignited (SI) engines. However, one aspect of ethanol is its lower energy content than gasoline (˜30%). The result is a reduction in the miles per gallon (mpg) for engines using ethanol. Promising strategies for mitigating the negative effect of reduced energy density of ethanol by leveraging the positive effect of high octane number are and are assisted with accurate, real-time estimates of the blend ratio of ethanol in a gasoline-ethanol blend for lean-burn gasoline engines. In one embodiment of the present invention, there is an algorithm for sensing in real-time the blend ratio, and making a change in one or more engine control schedules based on the estimated blend ratio.

Results from both the theoretical model as well as the experimental data presented in this work indicate that exhaust oxygen content, as measured by a commercial grade wideband O₂ sensor, coupled with knowledge of the mixture fraction, can be used to estimate the biodiesel (or ethanol) blend in a diesel (or gasoline) engine operating at steady-state conditions. Although what is shown and described herein is a wide band O₂ sensor, embodiments of the present invention are not so limited, and include those embodiments including any sensor that can provide a signal that corresponds to the free oxygen content of the exhaust gas. Furthermore, this estimation can be accomplished by an estimation algorithm with a simple form:

$${B}_{vol} \approx {C}_{1} ( \frac{{x}_{{O}_{2}}}{f} ) + {C}_{2} ( \frac{1}{f} ) + {C}_{3} \qquad ( A )$$

where Bᵥₒₗ is the volumetric blend fraction, χ_{O2} is the exhaust O₂ mole fraction, f is the mixture fraction, and C₁, C₂, and C₃ are constants. An estimator of this form, relying on the model for C₁, C₂, and C₃, may slightly mis-predict a biodiesel blend when applied to the experimental data. However, when a portion of the experimental data was used to derive “trained” values of the constants C₁, C₂, and C₃, the “trained” estimator predicted the blend correctly to within 4.2% for all four fuel blends tested. This indicates that the above estimator form can be used to estimate the biodiesel blend. However, yet other embodiments of the present invention are not so limited and are useful during transient operation.

In yet another embodiment of the present invention, the simplified estimation (A) algorithm shown above can be rewritten in terms of the mass flow rate of fuel and mass flow rate of air based on equation (B) herein to the following form:

$${B}_{vol} \approx {C}_{1} {x}_{{O}_{2}} ( \frac{{\dot{m}}_{air}}{{\dot{m}}_{fuel}} ) + {C}_{1} {x}_{{O}_{2}} + {C}_{2} ( \frac{{\dot{m}}_{air}}{{\dot{m}}_{fuel}} ) + {C}_{4} \\ where \\ {C}_{4} = {C}_{2} + {C}_{3} \qquad ( B )$$

In this equation above, it is seen that the volumetric blend fraction (B) can be calculated from knowledge from the mass airflow rate, mass fuel flow rate, and exhaust oxygen mole fraction. It can be seen that the blend fraction is linearly and proportionally related to oxygen mole fraction and air mass flow rate, and inversely proportional to mass fuel flow rate. In those engines having an electronic control module (ECM) that has real time values for airflow, fuel flow, and exhaust gas oxygen concentration, it is possible to use equation (B) to calculate in real time an estimate of the blend of the fuel being consumed by the engine. With knowledge of this blend ratio, it is possible for the ECM to modify the operation of various engine actuators by using the knowledge of the blend ratio in various look-up tables or functional algorithms that relate blend ratios to an engine control schedule.

Although what has been shown and described is an engine having an ECM that used equation (B) in its software, the present invention is not so limited. The present invention contemplates those embodiments in which equations such as (A) and (B), as well as others described herein, are utilized in the preparation of the ECM software. As one example, equations (A) or (B) can be utilized by a programmer during the formative stages of developing the program, such as before the creation of any source code or object code. In some embodiments, the various elements of equations (A) and (B) are manipulated to account for the manner and type of data acquisition by the ECM. Further, equations (A) or (B) can be manipulated jointly with other controlling equations within the software, and the resultant integrated equations may be manipulated to the point that equations (A) or (B) are no longer recognizable. In such embodiments, the inventive methods described herein are nonetheless are part of the ECM software.

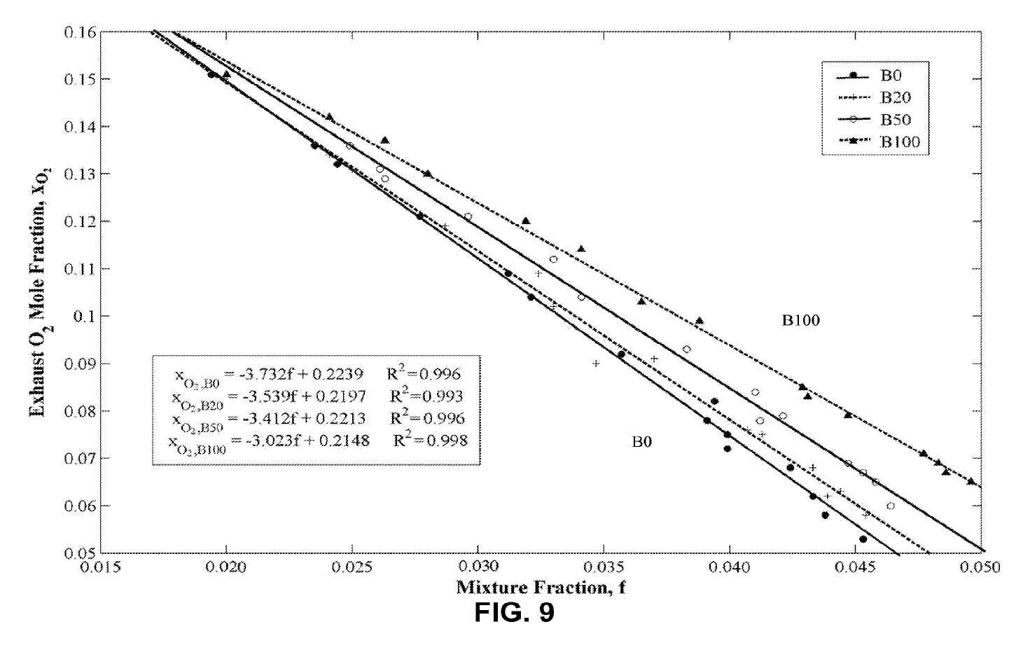

Experimental results indicate that, while the trained estimator algorithm works well at predicting the blend when applied to a number of data points, there can be variation on a data point by data point basis (See FIG. 9)—such as point-to-point noise. In an actual vehicle the fuel blend being delivered to the cylinders would typically take several minutes to change. Accordingly, for such implementations the estimator algorithm could be provided in a “continuously updating estimate” fashion, rather than an “instantaneous estimate” fashion. Such an implementation includes the instantaneous estimate, but also on the estimates over the course of the last few minutes, or other predetermined interval.

Alternatively or additionally, various noise handling techniques may be utilized to manage the blend ratio estimate and provide for a stable but responsive blend ratio estimate. For example, a low-pass filter having a time constant that allows the blend ratio to change within a small reasonable number of data points will allow the blend ratio to change due to real disturbances (e.g. an operator fills a fuel tank with a different fuel blend) while filtering out the point-to-point noise from the estimate. As yet another example, another smoothing algorithm includes collecting data points within a window of time and then eliminating one or more high values, and/or one or more low values. In still other embodiments, other noise/conditioning techniques may be used.

“Narrow-band” O₂ sensors have been widely used with spark-ignited (SI) gasoline engines since the late 1970's to measure oxygen concentrations in the exhaust steam. Wideband O₂ sensors, which enable accurate measurements under highly lean conditions, have also been widely used with production SI gasoline engines for several years. These wideband O₂ sensors are also suitable for use in diesel engines. In fact, a few diesel vehicles being produced today (such as the 2007 Dodge Ram pickups with the 6.7 liter Cummins ISB engine) already utilize wideband O₂ sensors to ensure optimal operation of advanced aftertreatment systems which are dependent on oxygen concentrations (such as lean NOₓ traps). In some embodiments, a wideband O₂ sensor is a practical sensor that, when coupled with knowledge of the mixture fraction and the approach presented in this work, may allow for the accurate estimation of biodiesel content in a diesel fuel blend, or ethanol content in a gasoline fuel blend, or a blend mixture of any two fuels having different stoichiometric mixture fractions. Examples herein utilize a wideband O₂ sensor for simplicity of the description. However, any sensor capable of providing an oxygen composition determination over the range of values experienced in an engine exhaust stream are contemplated herein, including, without limitation, an H₂O or CO₂ sensor.

Many of the chemical and physical properties of biofuels are different than those of conventional fuels. For biodiesel these properties include cetane number, density, lower heating value, viscosity, lubricity, and bulk modulus; and for ethanol, there can be differences in octane number and lower heating value compared to gasoline. Because these inputs to the combustion process are not the same as conventional fuels, it should not be surprising that the outputs of combustion (emissions, power/torque, etc.) are not the same for biofuels. Fortunately, research has shown that it might be possible to mitigate the negative aspects of biodiesel (higher NOₓ and reduced fuel economy) by active modulation of engine “actuators”, including injection timing, amount of exhaust gas recirculation (EGR), amount of turbo-charging, and injection pressure. Research has also shown that some aspects of ethanol (including lower fuel economy due to lower energy density) might be mitigated with intelligent control and use of turbo-charging and fuel injection modulation. All of these parameters, for both diesel and gasoline engines, can be controlled through the engine control module (ECM). To accommodate the differences in the combustion behavior of different fuels, various embodiments of the present invention include methods to estimate the properties of the fuel being injected into the cylinder.

As another example, consider again biodiesel. Recent research suggests that one of the reasons for the increased biodiesel NOₓ effect observed in modern diesel engine may be the effect that the lower energy content of the biodiesel. An electronically controlled engine may include a model for engine torque as a function of the injected fuel. Where the energy content of the fuel varies from the model assumptions, the actual generated torque of the engine is different from the modeled torque. Further, engines are typically calibrated with respect to fuel timing, EGR fraction, aftertreatment response, and/or other factors that vary with the energy and oxygen content of the fuel. Therefore, the use of a biodiesel blend can change the engine performance and/or emissions relative to design parameters where the engine controls do not have information about the fuel composition. One effect observed in engines burning biodiesel is a higher NOₓ output which may be mitigated with, in one example, a higher EGR fraction based on the biodiesel blend.

In modern diesel engines the ECM's decision making with regard to the engine actuation can be based on the measured engine speed and the estimated engine torque. The engine can be calibrated in such a way that for each torque speed condition, a predetermined amount of EGR is introduced, the ideal fresh air flow is achieved, etc. Torque, however may not be an ECM measured quantity on an engine. Instead, torque can be estimated based on the volume of fuel injected (assuming a certain energy content of the fuel). A biodiesel blend, however, contains less energy per unit volume than conventional diesel. Therefore, when a biodiesel blend is used, the torque estimate is higher than the actual torque, and the calibration may be thrown off (resulting in less than ideal EGR flow, for example). This effect of the lower energy content of biodiesel blends on the ECM's decision making is correctable, however, if there is a practical means of estimating what fuel blend is being used (and thus the actual energy content would be known and torque could be estimated more accurately).

Estimating the percentage of biofuels (for example, either biodiesel or ethanol) in the fuel blend will assist in allowing the ECM to maintain optimal engine performance across various fuel blends (B0 vs. B20 vs. B100, E0 vs. E85, etc.). One embodiment of the present invention pertains to a control algorithm that uses information from a is wideband oxygen (O₂) sensor in the exhaust stream, coupled with knowledge of fuel and air flow, to estimate the percentage of biodiesel or ethanol in the fuel blend. This technique permits real-time, on-board accommodation of variations in combustion behavior across different biodiesel blends in modern diesel engines, and different ethanol blends in gasoline engines. Creatively using a wideband O₂ sensor is attractive because they are an already established production sensor that, in some cases, is already installed on the vehicle.

Since biodiesel and ethanol are oxygenated fuels and conventional fuels are not, there are more oxygen atoms present in the cylinder prior to combustion for a given mixture fraction (mixture fraction is the mass fraction of the fuel-air mixture that is fuel). Therefore, since the hydrogen/carbon atom ratio for conventional and biofuels are similar, post combustion oxygen concentrations (oxygen left over after combustion) should be higher for biofuels. One hypothesis for this work is that the level of oxygen in the exhaust stream will be indicative of the percentage of biofuel in the fuel blend, with the highest oxygen concentration expected for B100 (E100) and the lowest for B0 (E0). This provides a basis for developing a two-input, one output biofuel blend estimation strategy utilizing a wideband O₂ sensor in the exhaust stream along with estimates of air and fuel flow. A block diagram of the proposed approach is shown in FIG. 2. Air, fuel, exhaust, and EGR flows have been assumed to be at steady-state. Some embodiments of the present invention pertain to control algorithms that account for engine transient operation. A transient estimator according to one embodiment of the present invention includes additional inputs such as engine speed, EGR valve position, etc. to be added.

In a conventional SI engine, an O₂ sensor is present so that the exhaust oxygen concentrations can be maintained in a narrow range where the air-fuel ratio is nearly stoichiometric (no excess fuel, no excess air). In diesel and next generation lean-burn gasoline engines, however, combustion is lean of stoichiometric (i.e. excess air is present), and the air-fuel ratio undergoes large fluctuations depending on operating conditions. The model which the proposed estimation strategy is based upon in one embodiment, assumes lean, complete combustion to major products for the purposes of predicting exhaust O₂ concentrations.

Combustion in diesel and lean-burn gasoline engines is significantly lean of stoichiometric and combustion inefficiency is ≦ about 2%, indicating substantially complete conversion of the fuel. Under these conditions, the global reaction of a generic oxygenated hydrocarbon fuel (CₙHₘOᵣ) with idealized air (O₂+εN₂) to major products (CO₂, H₂O, O₂, and N₂) is:

$${C}_{n} {H}_{m} {O}_{r} + λ ( n + \frac{m}{4} - \frac{r}{2} ) [ {O}_{2} + ε {N}_{2} + δ {H}_{2} O + ψ Ar ] -> n {CO}_{2} + ( \frac{m}{2} + λδ ( n + \frac{m}{4} - \frac{r}{2} ) ) {H}_{2} O + ( λ - 1 ) ( n + \frac{m}{4} - \frac{r}{2} ) {O}_{2} + λε ( n + \frac{m}{4} - \frac{r}{2} ) {N}_{2} + λψ ( n + \frac{m}{4} - \frac{r}{2} ) Ar \qquad ( 1 )$$

where n, m, and r are the number of carbon, hydrogen, and oxygen atoms in the fuel molecule, respectively. λ is the excess air factor=1/equivalence ratio=actual air to fuel ratio/stoichiometric air to fuel ratio, ε is the mole ratio of nitrogen to oxygen in air, δ is the mole ratio of water vapor to oxygen, and φ is the mole ratio of argon to oxygen in air.

The mixture fraction, f, is a function of the mass flow rate of air and the mass flow rate of fuel (both of which are typically controlled and estimated by the ECM in modern engines). The mixture fraction is related to the air to fuel ratio by:

$$f = \frac{{\dot{m}}_{fuel}}{{\dot{m}}_{fuel} + {\dot{m}}_{air}} = \frac{1}{1 + AFR} \qquad ( 2 )$$

The above definition can be used to define the excess air factor λ in terms of the mixture fraction f:

$$λ = ( \frac{1 - f}{f} ) \frac{( n α + m β + r γ )}{( n + \frac{m}{4} - \frac{r}{2} )} \qquad ( 3 )$$

where α, β, and γ are constants defined as:

$$α = \frac{{a}_{C}}{( 2 + δ ) {a}_{O} + 2 δ {a}_{H} + 2 ε {a}_{N} + ψ {a}_{Ar}} , \\ β = \frac{{a}_{H}}{( 2 + δ ) {a}_{O} + 2 δ {a}_{H} + 2 ε {a}_{N} + ψ {a}_{Ar}} , \\ γ = \frac{{a}_{O}}{( 2 + δ ) {a}_{O} + 2 δ {a}_{H} + 2 ε {a}_{N} + ψ {a}_{Ar}}$$

with a_{C}, a_{H}, a_{O}, and a_{N} representing the atomic masses of carbon, hydrogen, oxygen, and nitrogen, respectively.

Substituting (3) back into (1) yields (4), the global reaction in terms of the mixture fraction.

$${C}_{n} {H}_{m} {O}_{r} + ( \frac{1 - f}{f} ) ( n α + m β + r γ ) [ {O}_{2} + ε {N}_{2} + δ {H}_{2} O + ψ Ar ] \to n {CO}_{2} + ( δ ( \frac{1 - f}{f} ) ( n α + m β + r γ ) + \frac{m}{2} ) {H}_{2} O + ( ( \frac{1 - f}{f} ) ( n α + m β + r γ ) - n - \frac{m}{4} + \frac{r}{2} ) {O}_{2} + ε ( \frac{1 - f}{f} ) ( n α + m β + r γ ) {N}_{2} + ψ ( \frac{1 - f}{f} ) ( n α + m β + r γ ) Ar \qquad ( 4 )$$

Examination of (4) yields (5), the mole fraction of O₂ in the exhaust stream.

$${x}_{{O}_{2}} = \frac{( \frac{1 - f}{f} ) ( n α + m β + r γ ) - n - \frac{m}{4} + \frac{r}{2}}{( \frac{1 - f}{f} ) ( n α + m β + r γ ) ( ε + δ + ψ + 1 ) + \frac{m}{4} + \frac{r}{2}} \qquad ( 5 )$$

Equation (5) captures the dependence of exhaust O₂ levels on the fuel's molecular structure (via n, m, and r) and the proportions of air and fuel (via f) brought into the cylinder for lean, substantially complete combustion. Representative n, m, and r values for a biodiesel-diesel or ethanol-gasoline blends can be found via (6).

n=n_{A}+Bₘₒₗ(n_{B}−n_{A}),

m=m_{A}+Bₘₒₗ(m_{B}−m_{A})

r=r_{A}+Bₘₒₗ(r_{B}−r_{A}) (6)

where the subscripts A and B denote fuel A and fuel B, respectively. Bₘₒₗ represents the blend fraction on a molar basis (moles of fuel B per total moles of fuel). Typically, however, the blend is not known on a molar basis, but rather, on a volumetric basis (volume of fuel B per total volume of fuel). By definition, the molar and volumetric blends are related by:

$${B}_{mol} = \frac{{B}_{vol} {R}_{MW}}{{B}_{vol} {R}_{MW} + {R}_{p} ( 1 - {B}_{vol} )} \qquad ( 7 )$$

where Bᵥₒₗ is the blend fraction on a volumetric basis, and the MW and ρ terms represent molecular weight and density, respectively. Note that Bᵥₒₗ is the blend fraction (not the blend percentage). Bᵥₒₗ=0.1 means the blend is 10% fuel B by volume, etc.

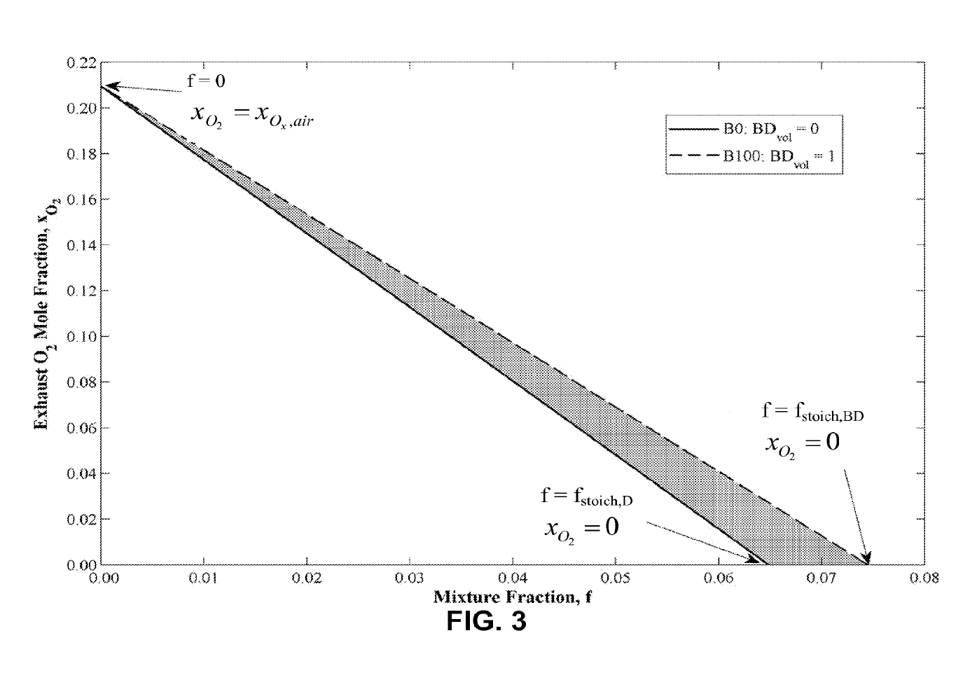

Equations (5), (6), and (7) allow for the prediction of exhaust oxygen levels as a function of mixture fraction and volumetric blend. As an example consider FIG. 3 which displays predicted exhaust O₂ mole fractions across all lean mixture fractions for conventional diesel (B0) and soy-based methyl ester biodiesel (B 100). The numeric values of the parameters used are given in Table I. Note that the shaded region represents the space in which all O₂ concentrations (regardless of blend) are predicted. O₂ concentrations are expected to be on the lower limit of the region for B0 and the upper limit of the region for B100. Note that predicted O₂ concentrations for B0 and B 100 converge at a mixture fraction of zero. This is because a mixture fraction of zero represents pure air (no fuel), thus the O₂ concentration is that of air (approximately 21%). Also note that when O₂ concentrations are zero, this represents stoichiometric conditions (all the oxygen in the air is consumed during combustion). The distinction between blends becomes more substantial as the mixture fraction increases because a greater percentage of fuel in present in the fuel-air mixture. This distinction between the O₂ levels between conventional diesel and biodiesel provides the basis for the estimation of the percentage of biodiesel in the fuel blend given knowledge of exhaust O₂ and the mixture fraction. Note that while (5) is clearly not linear with respect to mixture fraction, the relationship between O₂ and mixture fraction shown in FIG. 3 appears to be nearly linear.

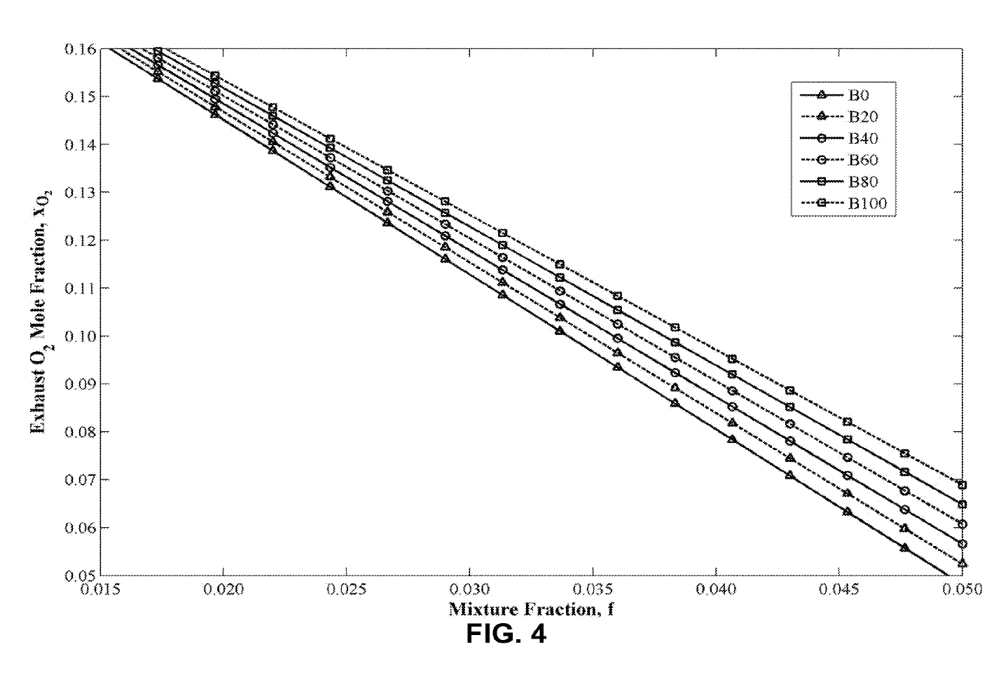

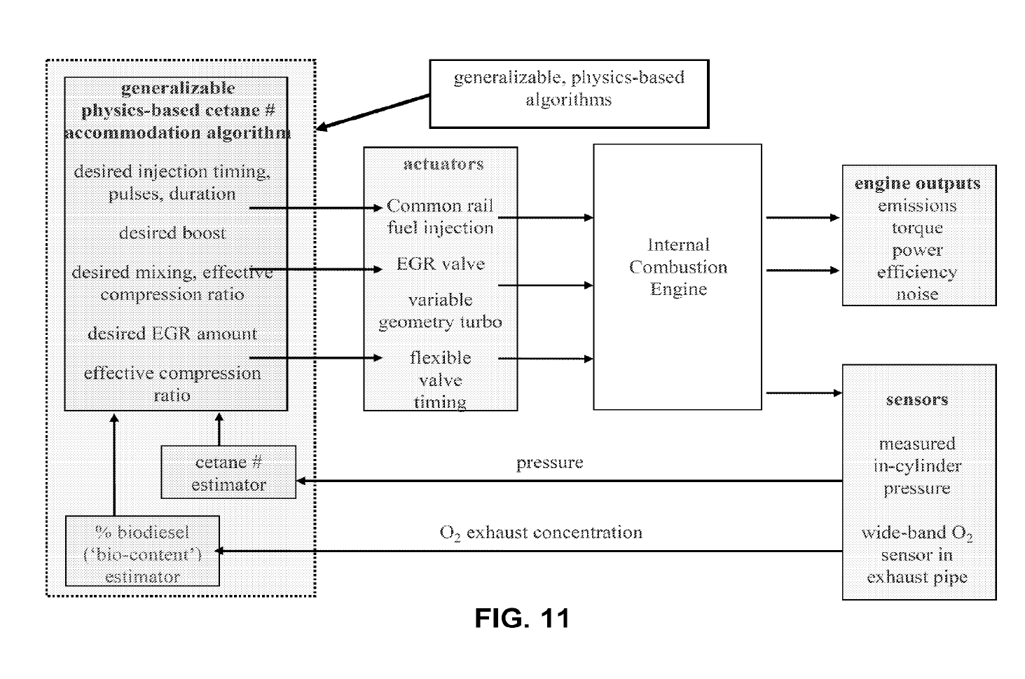

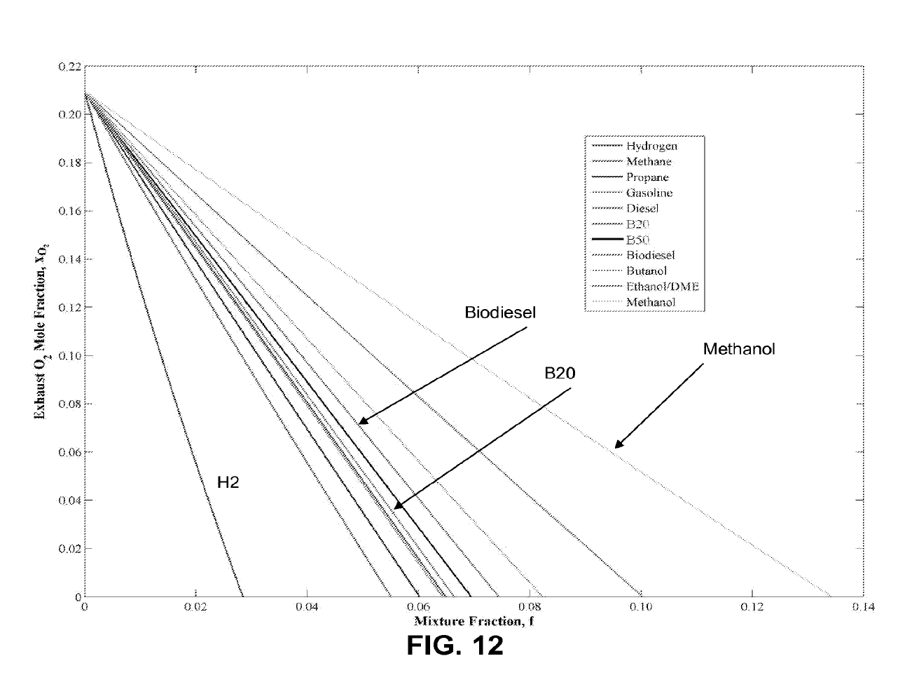

FIG. 4 displays predicted exhaust O₂ mole fractions across mixture fractions from 0.015 to 0.05 (air-fuel ratios from 65 to 19) for blends of soy-based biodiesel. FIG. 11 presents a simplified schematic representation of an engine model. FIG. 12 is a simplified block diagram of an engine control and simulation model according to one embodiment of the present invention. This region is of the greatest interest for combustion in diesel engines. The numeric values of the parameters used are given in Table I. Note that the blends of B20, B40, B60 and B80 appear to be equally spaced across the space between B0 and B100.

TABLE I
CONSTANTS USED IN MODEL

| Parameter | Symbol | Value | Units |
|---|---|---|---|
| atomic mass of carbon | a_{C} | 12.011 | kg/kmol |
| atomic mass of hydrogen | a_{H} | 1.0079 | kg/kmol |
| atomic mass of oxygen | a_{O} | 15.999 | kg/kmol |
| atomic mass of nitrogen | a_{N} | 14.007 | kg/kmol |
| C atoms per diesel | n_{D} | 14.01 | none |
| molecule | | | |
| H atoms per diesel | m_{D} | 25 | none |
| molecule | | | |
| O atoms per diesel | r_{D} | 0 | none |
| molecule | | | |
| C atoms per biodiesel | n_{B} | 18.82 | none |
| molecule | | | |
| H atoms per biodiesel | m_{B} | 34.53 | none |
| molecule | | | |
| O atoms per biodiesel | r_{B} | 2 | none |
| molecule | | | |
| mole ratio of N₂ to O₂ in air | ε | 3.728 | None |
| mole ratio of H₂0 to O₂ in | δ | 0.0446 | None |
| air | | | |
| mole ratio of Ar to O₂ in air | φ | 0.0551* | None |
| density of diesel | ρ_{D} | 855.9 | kg/m³ |
| density of biodiesel | ρ_{B} | 879.6 | kg/m³ |

*Equivalent to 40% relative humidity at 20° C.

One method of estimating biofuel blend levels given exhaust O₂ and mixture fraction information is to combine (5), (6), and (7) and solve for Bᵥₒₗ. The result is (8), which gives the volumetric biofuel blend fraction as an explicit function of mixture fraction and exhaust O₂ mole fraction (both measurable quantities).

$${B}_{vol} = {( 1 - \frac{{R}_{MW} ( {Nn}_{B} + {Mm}_{B} + {Rr}_{B} )}{{R}_{p} ( {Nn}_{A} + {Mm}_{A} + {Rr}_{A} )} )}^{- 1} = function ( {x}_{{O}_{2}} , f ) \\ where \\ N = ( \frac{1 - f}{f} ) α - ( \frac{1 - f}{f} ) α ( ε + δ + ψ + 1 ) {x}_{{O}_{2}} - 1 , \\ M = ( \frac{1 - f}{f} ) β - ( ( \frac{1 - f}{f} ) β ( ε + δ + ψ + 1 ) + \frac{1}{4} ) {x}_{{O}_{2}} - \frac{1}{4} , \\ R = ( \frac{1 - f}{f} ) γ - ( ( \frac{1 - f}{f} ) γ ( ε + δ + ψ + 1 ) + \frac{1}{2} ) {x}_{{O}_{2}} + \frac{1}{2} \qquad ( 8 )$$

FIG. 4 indicates that O₂ levels are approximately linear with respect to mixture fraction, that is:

x_{O}_{₂}≈a₁f+b₁ (9)

where b₁ is constant and a₁ is constant with respect to mixture fraction. Additionally, it appears that the slope of the lines in FIG. 4 is approximately linear with respect to the volumetric blend level, that is:

a₁≈a₂Bᵥₒₗ+b₂ (10)

where a₂ and b₂ are constants. These two approximations yield (11), a simplified form of (8) which indicates that the volumetric blend level is approximately equal to a constant times the quotient of O₂ mole fraction and mixture fraction, plus a constant times the reciprocal of mixture fraction, plus a third constant.

$${B}_{vol} \approx {C}_{1} ( \frac{{x}_{{O}_{2}}}{f} ) + {C}_{2} ( \frac{1}{f} ) + {C}_{3} \\ where \\ {C}_{1} = \frac{1}{{a}_{2}} , {C}_{2} = \frac{{b}_{1}}{{a}_{2}} , {C}_{3} = \frac{{b}_{2}}{{a}_{2}} \qquad ( 11 )$$

The values of the constants C₁, C₂, and C₃ which cause (11) to reflect (8) can be found by sampling (8) over the region of interest and using the least squares method, the solution of which is (12).

$$[ \begin{matrix}{C}_{1} \\ {C}_{2} \\ {C}_{3}\end{matrix} ] = {( {B}^{T} B )}^{- 1} {B}^{T} d \\ where \\ B = [ \begin{matrix}\frac{{x}_{{O}_{2} , 1}}{{f}_{1}} & \frac{1}{{f}_{1}} & 1 \\ \frac{{x}_{{O}_{2} , 2}}{{f}_{2}} & \frac{1}{{f}_{2}} & 1 \\ ⋮ & ⋮ & ⋮ \\ \frac{{x}_{{O}_{2} , k}}{{f}_{k}} & \frac{1}{{f}_{k}} & 1\end{matrix} ] , d = [ \begin{matrix}{B}_{vol , 1} \\ {B}_{vol , 2} \\ ⋮ \\ {B}_{vol , k}\end{matrix} ] \qquad ( 12 )$$

Each row of B and d (labeled 1, 2, . . . , k) represents one sample of (8). Using the numeric values given in Table I for biodiesel as an example, the best fit over the region where 0.015≦f≦0.050 and 0≦Bᵥₒₗ≦1 is:

$${B}_{vol , best fit} = 2.4122 ( \frac{{x}_{{O}_{2}}}{f} ) - 0.5000 ( \frac{1}{f} ) + 7.7859 \qquad ( 13 )$$

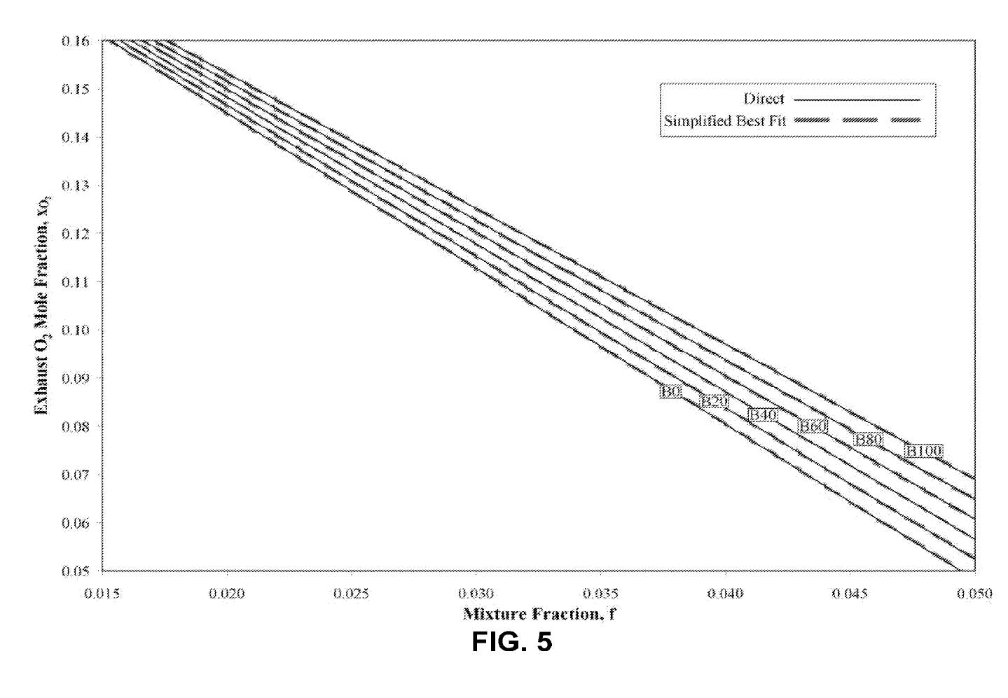

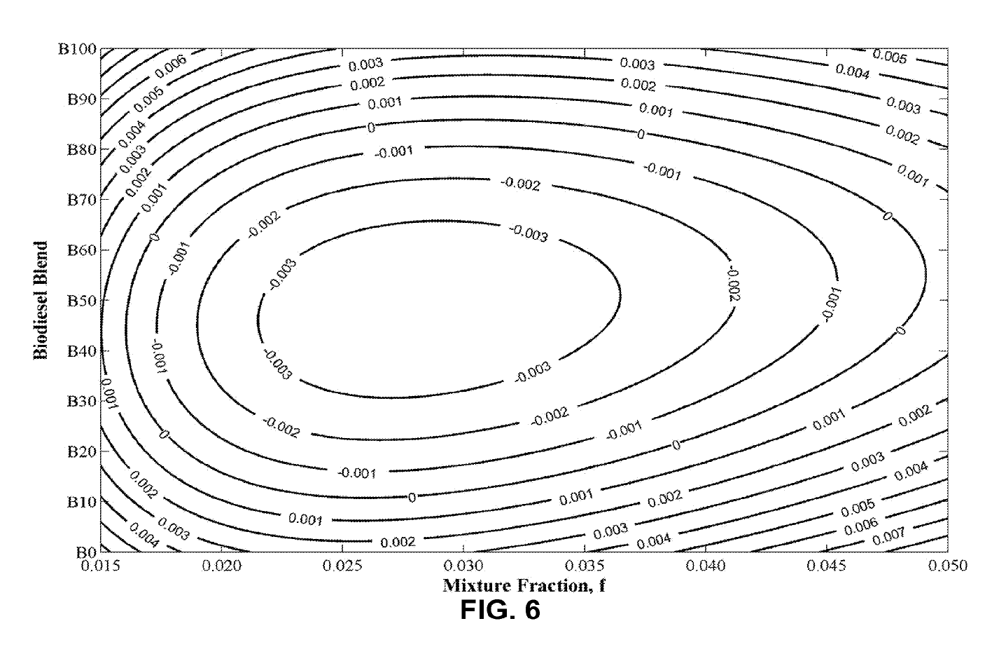

FIG. 5 displays exhaust O₂ mole fractions as predicted by the direct model as well as by the least squares best fit. The fit is nearly perfect. The contour plot in FIG. 6 shows that the difference in Bvol between (8) and (13) across this region is always less than 0.0095 (less than the difference between B99 and B 100). The coefficient of determination (R²) for this fit was also 0.99992, indicating an outstanding fit. This indicates that the complex equation (8) can be accurately captured by a much simpler and intuitive equation in the form of (11).

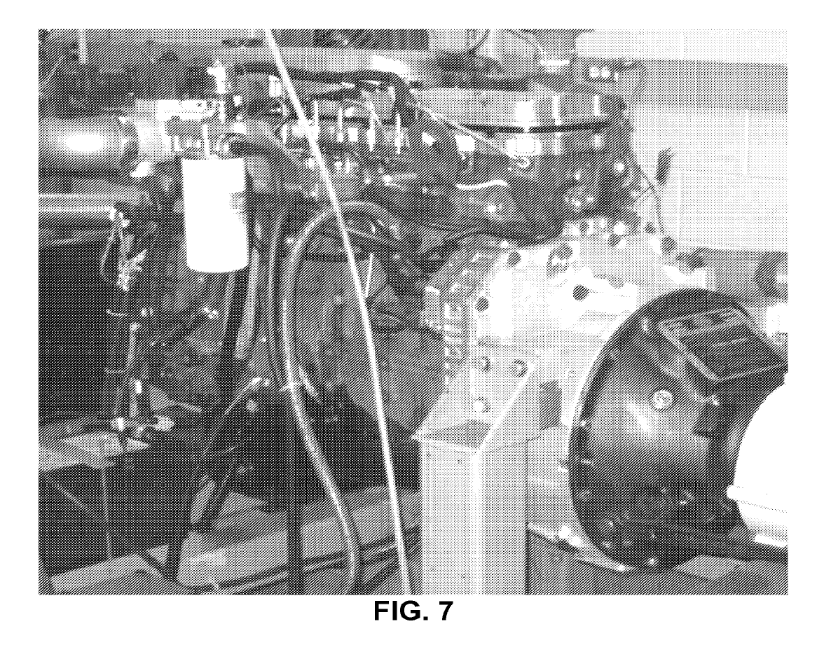

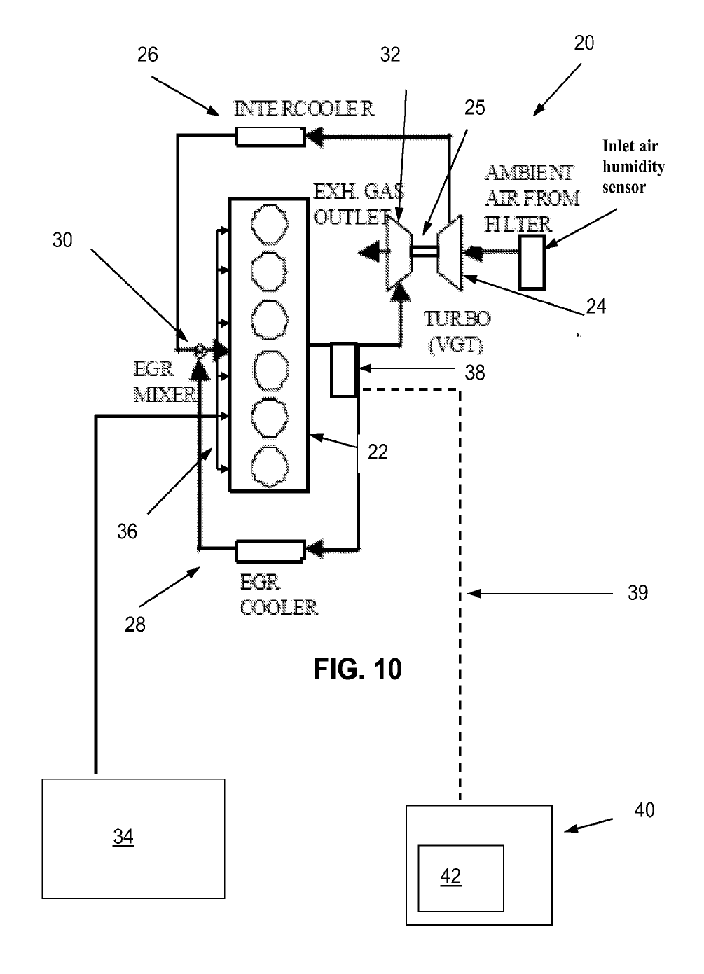

The engine used for this work (shown photographically in FIG. 7 and schematically in FIG. 10) was a 325-hp inline 6-cylinder 2007 Cummins 6.7 liter 24-valve ISB series engine with a variable geometry turbocharger (VGT), common rail fuel injection, and cooled EGR. Intake air flow was measured via a laminar flow element. Fuel consumption was determined gravimetrically. The wideband oxygen sensor used was a commercial grade Bosch LSU 4.9 (Bosch #0258017025).

Referring to FIG. 10, there is shown an internal combustion engine 20 according to one embodiment of the present invention. Engine 20 includes a power unit 22 that combusts fuel and air to produce both usable power and waste heat. In one embodiment, power unit 22 is a reciprocating, piston-in-cylinder compression ignition engine. In yet another embodiment, power unit 22 is a spark ignition, piston-in-cylinder engine. In yet other embodiments, power unit 22 can be any type of internal combustion engine, including those referred to as Wankel engines, and further including those based on the Brayton cycle.

Power unit 22 receives ambient air in one embodiment from a compressor 24 of a turbocharger 25. The compressed air is reduced in temperature by an intercooler 26, and presented to a mixing device 30. Also being provided to mixing device 30 is exhaust gas from power unit 22, which preferably has been cooled by an EGR cooler 28. Exhaust gas from power unit 22 is also used as a source of energy for a turbine 32 of turbocharger 25 that is mechanically coupled to compressor 24. The output of mixing device 30 is presented to combustion chambers within power unit 22, where it is combusted with a source 34 of mixed fuel, the latter being injected by a fuel injector assembly 36.

The combusted exhaust gases are expelled from the combustion chambers, and the expelled gas is in fluid communication with means 38 for sensing free oxygen. In one embodiment, means 38 is a wide-band O2 sensor. A signal 39 corresponding to the free oxygen content of the combusted exhaust gases is provided by sensor 38 to an electronic control module 40. ECM 40 includes software 42 that receives various state parameters from engine 20, including signal 39. Software 42 includes an algorithm that uses the operational state of the engine 20 to determine the blend of fuels (in one embodiment, one fuel being oxygenated and the other fuel being non-oxygenated) of source 34, and controls the operation of engine 20 based on the estimate of the blend of fuels in source 34.

Four fuels blends were tested: B0, B20, B50, and B100. The B0 fuel used was 2007 Emission Certification Ultra Low Sulfur Diesel Fuel. The B100 used was soy methyl ester biodiesel produced by Chevron Phillips. The B20 and B50 fuel blends were produced by mixing the B0 and

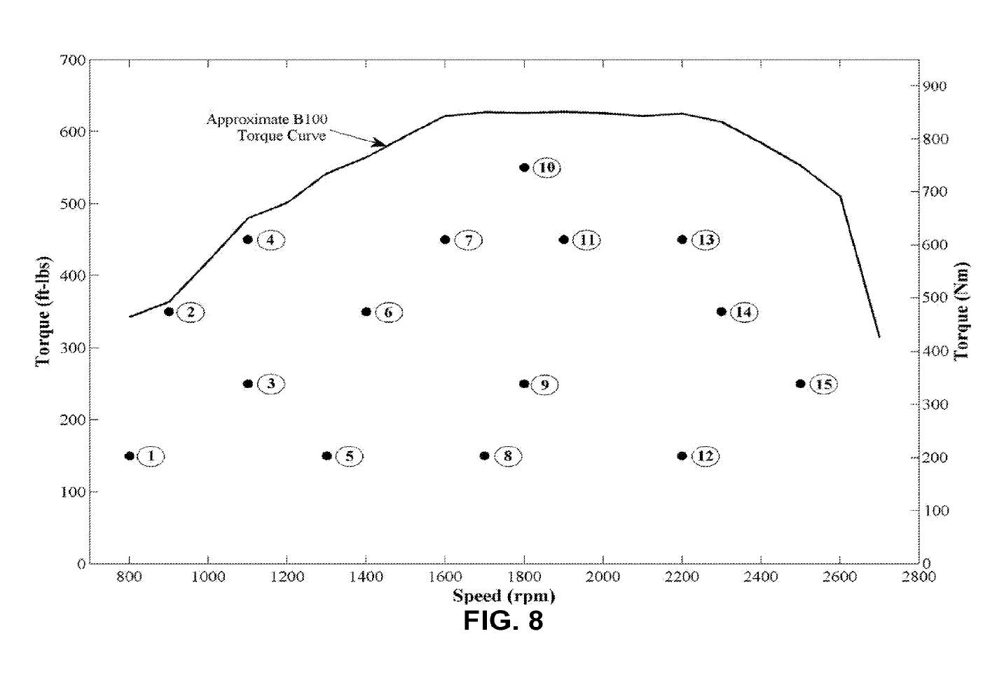

B 100 fuels on a volumetric basis. For each fuel blend, the engine was operated at 15 steady-state torque-speed points (Shown in FIG. 8 and listed in Table II). The engine was allowed to stabilize at each torque-speed point and then data collection took place over 5 minute time periods of steady-state operation.

TABLE II
EXPERIMENTAL DATA COLLECTION TORQUE-SPEED POINTS

| Point | | | |
|---|---|---|---|
| # | Speed | Torque | Power |
| — | rpm | ft-lbs (Nm) | hp (kW) |
| 1 | 800 | 150(203.4) | 22.8(17.0) |
| 2 | 900 | 350(474.5) | 60.0(44.7) |
| 3 | 1100 | 250(339.0) | 52.4(39.0) |
| 4 | 1100 | 450(610.1) | 94.2(70.3) |
| 5 | 1300 | 150(203.4) | 37.1(27.7) |
| 6 | 1400 | 350(474.5) | 93.3(69.6) |
| 7 | 1600 | 450(610.1) | 137.1(102.2) |
| 8 | 1700 | 150(203.4) | 48.5(36.2) |
| 9 | 1800 | 250(339.0) | 85.7(63.9) |
| 10 | 1800 | 550(745.7) | 188.5(140.6) |
| 11 | 1900 | 450(610.1) | 162.8(121.4) |
| 12 | 2200 | 150(203.4) | 62.8(46.9) |
| 13 | 2200 | 450(610.1) | 188.5(140.6) |
| 14 | 2300 | 350(474.5) | 153.3(114.3) |
| 15 | 2500 | 250(339.0) | 119.0(88.7) |

The experimental data collected for the B0, B20, B50, and B100 fuel blends is shown in Tables III, IV, V, and VI, respectively. The air-fuel ratio and mixture fraction values shown were calculated from the air and fuel flow values.

TABLE III
B0 EXPERIMENTAL DATA

| | Air | Fuel | | | |
|---|---|---|---|---|---|
| | Flow | Flow | Exhaust | Air-Fuel | Mixture |
| Pt. # | Rate | Rate | O₂ | Ratio | Fraction |
| — | kg/min | kg/min | mol/mol | — | — |
| 1 | 1.63 | 0.067 | 0.082 | 24.4 | 0.0394 |
| 2 | 3.67 | 0.162 | 0068 | 22.6 | 0.0424 |
| 3 | 4.87 | 0.139 | 0.121 | 35.1 | 0.0277 |
| 4 | 5.60 | 0.254 | 0.062 | 22.1 | 0.0433 |
| 5 | 5.42 | 0.107 | 0.151 | 50.5 | 0.0194 |
| 6 | 6.09 | 0.253 | 0.072 | 24.1 | 0.0399 |
| 7 | 7.94 | 0.376 | 0.053 | 21.1 | 0.0453 |
| 8 | 6.03 | 0.151 | 0.132 | 40.0 | 0.0244 |
| 9 | 7.41 | 0.246 | 0.104 | 30.2 | 0.0321 |
| 10 | 11.34 | 0.520 | 0.058 | 21.8 | 0.0438 |
| 11 | 10.88 | 0.452 | 0.075 | 24.1 | 0.0399 |
| 12 | 9.11 | 0.219 | 0.136 | 41.5 | 0.0235 |
| 13 | 13.23 | 0.538 | 0.078 | 24.6 | 0.0391 |
| 14 | 12.02 | 0.445 | 0.092 | 27.0 | 0.0357 |
| 15 | 11.59 | 0.374 | 0.109 | 31.0 | 0.0312 |

TABLE IV
B20 EXPERIMENTAL DATA

| | Air | Fuel | | | |
|---|---|---|---|---|---|
| | Flow | Flow | Exhaust | Air-Fuel | Mixture |
| Pt. # | Rate | Rate | O₂ | Ratio | Fraction |
| — | kg/min | kg/min | mol/mol | — | — |
| 1 | 1.90 | 0.068 | 0.090 | 27.8 | 0.0347 |
| 2 | 3.67 | 0.166 | 0.068 | 22.1 | 0.0433 |
| 3 | 4.87 | 0.144 | 0.196 | 33.9 | 0.0287 |
| 4 | 5.60 | 0.260 | 0.063 | 21.5 | 0.0444 |
| 5 | 5.44 | 0.111 | 0.150 | 49.2 | 0.0199 |
| 6 | 6.10 | 0.262 | 0.075 | 23.2 | 0.0413 |
| 7 | 8.21 | 0.390 | 0.058 | 21.0 | 0.0454 |
| 8 | 6.01 | 0.154 | 0.131 | 38.9 | 0.0250 |
| 9 | 7.38 | 0.252 | 0.102 | 29.3 | 0.0330 |
| 10 | 11.47 | 0.526 | 0.062 | 21.8 | 0.0439 |
| 11 | 10.89 | 0.462 | 0.076 | 23.6 | 0.0407 |
| 12 | 9.07 | 0.224 | 0.134 | 40.6 | 0.0241 |
| 13 | 13.05 | 0.554 | 0.076 | 23.5 | 0.0407 |
| 14 | 11.92 | 0.458 | 0.091 | 26.0 | 0.0370 |
| 15 | 11.49 | 0.385 | 0.109 | 29.8 | 0.0324 |

TABLE V
B50 EXPERIMENTAL DATA

| | Air | Fuel | | | |
|---|---|---|---|---|---|
| | Flow | Flow | Exhaust | Air-Fuel | Mixture |
| Pt. # | Rate | Rate | O₂ | Ratio | Fraction |
| — | kg/min | kg/min | mol/mol | — | — |
| 1 | 2.68 | 0.072 | 0.129 | 37.1 | 0.0263 |
| 2 | 3.69 | 0.173 | 0.069 | 21.4 | 0.0447 |
| 3 | 4.89 | 0.149 | 0.121 | 32.8 | 0.0296 |
| 4 | 5.64 | 0.271 | 0.065 | 20.9 | 0.0458 |
| 5 | 5.51 | 0.115 | 0.152 | 48.1 | 0.0204 |
| 6 | 6.37 | 0.274 | 0.078 | 23.2 | 0.0412 |
| 7 | 8.39 | 0.408 | 0.060 | 20.5 | 0.0464 |
| 8 | 6.05 | 0.162 | 0.131 | 37.4 | 0.0261 |
| 9 | 7.45 | 0.263 | 0.104 | 28.3 | 0.0341 |
| 10 | 11.47 | 0.544 | 0.067 | 21.1 | 0.0453 |
| 11 | 10.98 | 0.482 | 0.079 | 22.8 | 0.0421 |
| 12 | 9.15 | 0.234 | 0.136 | 39.2 | 0.0249 |
| 13 | 13.33 | 0.570 | 0.084 | 23.4 | 0.0410 |
| 14 | 11.92 | 0.474 | 0.093 | 25.1 | 0.0383 |
| 15 | 11.53 | 0.394 | 0.112 | 29.3 | 0.0330 |

TABLE VI
B100 EXPERIMENTAL DATA

| | Air | Fuel | | | |
|---|---|---|---|---|---|
| | Flow | Flow | Exhaust | Air-Fuel | Mixture |
| Pt. # | Rate | Rate | O₂ | Ratio | Fraction |
| — | kg/min | kg/min | mol/mol | — | — |
| 1 | 3.18 | 0.079 | 0.142 | 40.5 | 0.0241 |
| 2 | 3.71 | 0.186 | 0.071 | 20.0 | 0.0477 |
| 3 | 4.89 | 0.161 | 0.120 | 30.4 | 0.0319 |
| 4 | 5.55 | 0.289 | 0.065 | 19.2 | 0.0496 |
| 5 | 5.61 | 0.115 | 0.151 | 48.9 | 0.0200 |
| 6 | 6.56 | 0.296 | 0.083 | 22.2 | 0.0431 |
| 7 | 8.55 | 0.437 | 0.067 | 19.6 | 0.0486 |
| 8 | 5.98 | 0.173 | 0.130 | 34.7 | 0.0280 |
| 9 | 7.47 | 0.283 | 0.103 | 26.4 | 0.0365 |
| 10 | 11.63 | 0.591 | 0.069 | 19.7 | 0.0483 |
| 11 | 11.02 | 0.516 | 0.079 | 21.4 | 0.0447 |
| 12 | 9.18 | 0.248 | 0.137 | 37.0 | 0.0263 |
| 13 | 13.64 | 0.611 | 0.085 | 22.3 | 0.0429 |
| 14 | 12.55 | 0.506 | 0.099 | 24.8 | 0.0388 |
| 15 | 11.90 | 0.420 | 0.114 | 28.3 | 0.0341 |

TABLE VII
ESTIMATOR RESULTS

| Actual | Estimated |
|---|---|
| Blend | Blend |
| B0 | B-0.6 |
| B20 | B14.6 |
| B50 | B55.8 |
| B100 | B101.2 |
| B0 | B-18.5 |
| B20 | B-2.9 |
| B50 | B38.4 |
| B100 | B84.4 |

FIG. 9 displays the experimental data collected for all four blends. The least squares best fit lines for all four fuel blends are also shown. The coefficients of determination (R²) for all four best fit lines exceed 0.99, supporting the assumption, (9), made in developing the simplified model that O₂ is essentially linear with respect to mixture fraction. The B50 data also falls approximately halfway between the B0 and B100 data and the B20 data is slightly closer to B0 than B50. This supports the assumption, (10), made in developing the simplified model that the slope of the lines is relatively linear with respect to the volumetric biodiesel blend fraction.

If (13), which is based on the theoretical model, is used to estimate the blend, it estimates the blend within 6% of the true blend as can be seen in Table VII. The values shown are the mean estimated value for all 15 points of each blend. Alternatively, the estimator constants can be determined by “training” the estimator in the form of (11) with a portion of the experimental data (rather than the theoretical model).

Some embodiments of the present invention include using a “trained” estimator that has been made specific based on data collected from one or more engines. However, other embodiments of the present invention include the use of the untrained, theoretical model.

Here the approach has been validated for biocontent estimation in biodiesel-diesel fuel blends, though as noted previously it is also applicable to ethanol-gasoline blends as well. It is anticipated that even better accuracy would be possible with ethanol blends because ethanol has a much higher level of oxygenation than biodiesel, and there for it would be easier to distinguish ethanol from gasoline.

The estimation methods presented herein have application outside biodiesel blends in diesel engines and ethanol blends in lean-burn gasoline engines. The approach should be of use in any application where blends of two fuels of differing stoichiometric mixture fraction being combusted in such a manner that the assumption of lean, essentially complete combustion in idealized air to form major products is a reasonably good assumption. Examples include ethanol diesel blends in diesel engines, ethanol gasoline blends in lean burn SI or HCCI/PCCI engines, as well as oxygenated fuel blends in non-automotive engines, such as gas turbine engines. It is anticipated that more resolution would be possible with ethanol blends than biodiesel blends because ethanol has a much higher level of oxygenation. FIG. 13 provides data representative of various fuels that can be blended, such that the blend ratio can be estimated by the algorithms and apparatus presented herein.

An exemplary embodiment is a method including providing an internal combustion engine having a fuel flow, an airflow, and an exhaust flow. The fuel flow includes fuel from a fuel source, where the fuel source is a binary fuel mixture or where the fuel source may at least intermittently include a binary fuel mixture. For example, the fuel source may include a diesel/bio-diesel mixture, a gas/ethanol mixture, or any other fuel source that includes two fuels having different stoichiometric ratios of fuel to available oxygen. The fuel source may be a fuel tank associated with an engine that is capable of burning fuels that have varying stoichiometric ratios, for example an engine that is presented as being capable of burning gasoline, ethanol, and mixtures thereof.

The method further includes providing a wide-band oxygen sensor disposed in the exhaust, where the wide-band oxygen sensor is capable of providing a variable response to an oxygen content of the exhaust flow. The wide-band oxygen sensor may provide a voltage or other electronic value in response to the oxygen content, and/or the wide-band oxygen sensor may provide a network or datalink communication indicative of an oxygen content of the exhaust flow. The method further includes operating the internal combustion engine, and interpreting an amount of the fuel flow and an amount of the airflow. Interpreting the fuel flow and airflow includes receiving a value of the fuel flow and airflow by any method known in the art, including at least reading values from a memory location on a computer readable medium, receiving electronic values, datalink, or network communications that are indicative of the fuel flow and airflow, and/or calculating the airflow or fuel flow from other values measured or calculated in the system.

The method further includes measuring an oxygen content of the exhaust flow with the wide-band oxygen sensor, and determining the blend fraction of the fuel flow in response to the amount of the fuel flow, the amount of the airflow, and the oxygen content of the exhaust flow. The determining the oxygen content includes, without limitation, performing calculations consistent with any description herein, and/or looking up values from a table stored in a computer memory and constructed according to any principles described herein. In certain embodiments, the method further includes determining the oxygen mole fraction of the exhaust flow in response to the oxygen content of the exhaust flow, and determining the composition of the fuel flow according to the equation:

$${VF}_{1} = \frac{100}{{f}_{sF 1} - {f}_{sF 2}} ( \frac{{x}_{{O}_{2} Air} \times {f}_{current}}{{x}_{{O}_{2} Air} - {x}_{{O}_{2} Exhaust}} - {f}_{sF 2} ) .$$

In the listed equation, VF₁ is a volumetric fraction of a first fuel in the binary fuel mixture, f_{sF1} is a stoichiometric mixture fraction for the first fuel, f_{sF2} is a stoichiometric mixture fraction for a second fuel in the binary fuel mixture, x_{O}_{₂}_{Air} is an oxygen mole fraction of air, f_{current} is a presently determined mixture fraction, and x_{O}_{₂}_{Exhaust} is the oxygen mole fraction of the exhaust flow. The f_{current} may be determined according to parameters ordinarily measured during the control of an electronic engine, or may be published as a readable parameter (e.g. in a memory location, on a network, and/or as a datalink parameter) by an engine controller. The engine controller may perform certain operations of the method, and/or certain operations of the method may be performed in one or more separate controllers, in “smart” sensors, or in other devices capable of providing calculated parameters.

In certain embodiments, the method further includes modifying an engine operating parameter in response to the oxygen content of the fuel flow. The modifying the engine operating parameter includes any operations understood in the art that may be performed in response to a fuel composition determination. Non-limiting examples of modified engine operating parameters include modifying an exhaust gas recirculation (EGR) flow rate, modifying the EGR fraction target, modifying a fuel injection pressure, modifying a fuel injection timing, modifying a torque rating of the engine, and/or modifying an emissions operating mode of the engine. Generally, but without limitation, modified engine behaviors will respond to reduce NOₓ production (e.g. higher EGR fraction, higher fuel injection pressure, relatively retarded fuel timing, lower maximum torque rating) when the fuel source includes a higher fraction of oxygenated fuel. However, any defined responses may be implemented, for example providing a different emissions schedule in response to legislated emissions based on fuel type, responses defined by a fleet owner according to the fuel type, or any other responses understood in the art. The modifying the emissions operating mode of the engine includes, without limitation, enabling or disabling certain emissions affecting features, changing an emissions target, providing an output parameter indicative of the composition of the fuel source, providing a compliance value, providing a fault value, and/or enabling or disabling an auxiliary emission control device (AECD).

Still another embodiment comprises: an internal combustion engine including an air intake to provide an airflow; a fuel source to provide a fuel flow to the engine to mix with the airflow for combustion by the engine, the engine producing an exhaust flow from the combustion; a wide-band oxygen sensor disposed in the exhaust flow to providing a signal representative of oxygen content; and a controller responsive to the signal, an amount of the fuel flow and an amount of the airflow to determine the blend fraction of a mixed fuel in the fuel flow.

Still a further embodiment is directed to an apparatus including an internal combustion engine having a fuel flow, an airflow, and an exhaust flow; means for providing the fuel flow as a binary fuel mixture or where the fuel source may at least intermittently include a binary fuel mixture; means for measuring oxygen content in the exhaust flow; an means for determining blend fraction of a mixed fuel in the fuel flow as a function of the oxygen content in the exhaust flow, an amount of fuel flow, and an amount of airflow. One aspect of the present invention pertains to a method for controlling an internal combustion engine including an oxygen sensor, an electronic controller, and a software algorithm for operating the engine according to the oxygen content of the fuel. Yet other embodiments include operating the engine by the algorithm with the controller. Still other embodiments include calculating a first number corresponding to the fuel flow rate during operating and calculating a second number corresponding to the airflow rate during operating. Still further embodiments include measuring the oxygen content of the exhaust gas.

Another aspect of the present invention pertains to a method for analyzing fuel combusted in an internal combustion engine. Other embodiments include providing an internal combustion engine and a mixed fuel. Further embodiments include combusting the mixed fuel with air in the engine, calculating a flow rate of mixed fuel and a flow rate of air during combusting, calculating the free oxygen content, and calculating a number corresponding to the ratio of the first fuel to the second fuel.

Yet another aspect of the present invention pertains to a method of controlling an internal combustion engine. Another aspect includes providing an internal combustion engine and a mixed fuel. Still further embodiments include operating the engine with the mixed fuel, calculating the flow rate of fuel into the engine, calculating the flow rate of air into the engine, measuring the free oxygen content of the exhaust gas from the engine, and calculating the ratio of the first composition to the second composition from the fuel flow rate, air flow rate, and oxygen content and using the ratio to modify said operating the engine.

While the inventions have been illustrated and described in detail in the drawings and foregoing description, the same is to be considered as illustrative and not restrictive in character, it being understood that only the preferred embodiment has been shown and described and that all changes and modifications that come within the spirit of the inventions are desired to be protected.

What is claimed is:

1. An apparatus comprising:
an internal combustion engine including an air intake to provide an airflow;
a fuel source to provide a fuel flow to the engine to mix with the airflow for combustion by the engine, the engine producing an exhaust flow from the combustion;
a wide-band oxygen sensor disposed in the exhaust flow for providing a signal representative of free oxygen content; and
a controller responsive to the signal, an amount of the fuel flow, and an amount of the airflow to determine oxygen content of fuel in the fuel flow.

2. The apparatus of claim 1, wherein said controller includes operating logic defining an estimator to determine the oxygen content of the fuel as a function of the signal, the amount of the fuel flow, and the amount of the airflow.

3. The apparatus of claim 1, wherein said controller includes memory and the operating logic is at least partially in the form of programming instructions executable by the controller, the programming instructions being stored in the memory.

4. The apparatus of claim 1, wherein said controller includes means for selectively providing exhaust gas recirculation in response to the oxygen content of the fuel.

5. The apparatus of claim 1, wherein said controller includes means for selectively adjusting an aftertreatment subsystem in response to the oxygen content of the fuel.

6. The apparatus of claim 1 wherein the fuel comprises a mixture of a first composition which includes oxygen and a second composition which substantially does not include oxygen.

7. The apparatus of claim 6 wherein the first composition is one of biodiesel or ethanol and the second composition is one of diesel fuel or gasoline.

8. The apparatus of claim 6 wherein the first composition is one of ethanol, methanol, dimethyl ether or biodiesel fuel.

9. The apparatus of claim 1 wherein said controller operates said engine with an air to fuel ratio that includes less than a stoichiometric amount of fuel.

10. A method of operating an internal combustion engine, comprising:
providing an internal combustion engine, and a mixed fuel including a first fuel which includes oxygen and a second fuel which substantially does not include oxygen;
operating the engine with the mixed fuel;
calculating the flow rate of fuel into the engine during said operating;
calculating the flow rate of air into the engine during said operating;
measuring the free oxygen content of the exhaust gas from the engine; and
interpreting the ratio of the first fuel to the second fuel from the fuel flow rate, air flow rate, and oxygen content.

11. The method of claim 10 which further comprises using the ratio to control the engine.

12. The method of claim 10, which further comprises modifying an engine operating parameter in response to the oxygen content of the fuel flow.

13. The method of claim 12, wherein said modifying comprises at least one of:
modifying an exhaust gas recirculation flow rate;
modifying an exhaust gas recirculation fraction target;
modifying a fuel injection pressure;
modifying a fuel injection timing;
modifying a torque rating of the engine; or
modifying an emissions operating mode of the engine.

14. The method of claim 10, which further comprises determining the oxygen content of the first fuel.

15. The method of claim 10, which further comprises determining an oxygen mole fraction of the exhaust flow in response to the oxygen content of the exhaust flow, and determining the composition of the fuel flow according to the equation:
$${VF}_{1} = \frac{100}{{f}_{sF 1} - {f}_{sF 2}} ( \frac{{x}_{{O}_{2} Air} \times {f}_{current}}{{x}_{{O}_{2} Air} - {x}_{{O}_{2} Exhaust}} - {f}_{sF 2} ) ,$$
wherein:
VF₁ is a volumetric fraction of a first fuel in a binary fuel mixture, f_{sF1} is a stoichiometric mixture fraction for the first fuel, f_{sF2} is a stoichiometric mixture fraction for a second fuel in the binary fuel mixture, x_{O}_{₂}_{Air} is an oxygen mole fraction of air, f_{current} is a presently determined mixture fraction, and x_{O}_{₂}_{Exhaust} is the oxygen mole fraction of the exhaust flow.

16. The method of claim 10 wherein the internal combustion engine is a compression ignition engine, and the second fuel is diesel fuel.

17. The method of claim 10 wherein the internal combustion engine is a spark ignition engine, and the second fuel is gasoline.

18. The method of claim 10 wherein the first composition is one of ethanol, methanol, dimethyl ether or biodiesel.

19. The method of claim 10 wherein said operating is with an air to fuel ratio that includes less than a stoichiometric amount of fuel.

20. A method of operating an internal combustion engine, comprising:
providing an internal combustion engine,
providing a first mixed fuel having a first predetermined mixture ratio of a first hydrocarbon fuel having a first molar quantity of oxygen mixed with a second hydrocarbon fuel having a second molar quantity of oxygen,
providing a second mixed fuel having a second predetermined mixture ratio of the first hydrocarbon fuel mixed with the second hydrocarbon fuel, the first mixture ratio being different than the second mixture ratio, and
providing a general relationship of fuel mixture ratio to the free oxygen content of the engine exhaust gas and also to at least one of the engine airflow rate or the engine fuel flow rate,
operating the engine with the first mixed fuel and measuring first data during said first operating including the free oxygen of the exhaust gas and the one of airflow rate or fuel flow rate;
operating the engine with the second mixed fuel and measuring second data during said second operating including the free oxygen of the exhaust gas and one of airflow rate or fuel flow rate; and
modifying the general relationship with the first data and the second data to a specific relationship.

21. The method of claim 20 wherein the engine is a first specific engine chosen from a family of similar engines, and which further comprises controlling a plurality of engines chosen from the family with an algorithm using the specific relationship.

22. The method of claim 20 wherein said providing includes a plurality of programmable electronic control modules each capable of controlling an internal combustion engine, and which further comprises programming the modules with software coding corresponding to the specific relationship.

23. The method of claim 20 wherein said providing includes an electronic control module having software, and which further comprises controlling the engine by the electronic control module with software coding corresponding to the specific relationship.

24. The method of claim 20 wherein said modifying includes preparing a tabular relationship of fuel mixture ratio to the free oxygen content.

25. The method of claim 20 wherein said modifying includes preparing a functional relationship of fuel mixture ratio to the free oxygen content.

26. The method of claim 20 wherein the general relationship includes a proportionality constant relating the fuel mixture ratio, the free oxygen content, and the one of airflow rate or fuel flow rate.

27. The method of claim 26 wherein said modifying includes assigning a number to the proportionality constant.

28. The method of claim 20 wherein the general relationship includes a mathematical combustion model having a term corresponding to free oxygen in the combustion products.

29. The method of claim 28 wherein said modifying includes changing the term.

30. The method of claim 20 wherein the general relationship includes a mathematical combustion model having terms corresponding to each of the free oxygen in the combustion products, the airflow rate, and the fuel flow rate.

31. The method of claim 20 wherein the general relationship includes at least 3 coefficients and said modifying includes assigning a value to each of the coefficients.

32. The method of claim 20 wherein one of the first or second molar quantities of oxygen is about zero.

33. The method of claim 20 wherein the engine is a compression ignition engine.

34. The method of claim 33 wherein one of the first hydrocarbon fuel or the second hydrocarbon fuel is diesel fuel.

35. The method of claim 34 wherein the other of the first hydrocarbon fuel or the second hydrocarbon fuel is a biodiesel fuel.

36. The method of claim 20 wherein the engine is a spark ignition engine.

37. The method of claim 36 wherein one of the first hydrocarbon fuel or the second hydrocarbon fuel is gasoline.

38. The method of claim 37 wherein the other of the first hydrocarbon fuel or the second hydrocarbon fuel is an alcohol.

39. A method of analyzing fuel combusted in an engine, comprising:
providing an internal combustion engine, a first mixed fuel having a first mixture ratio of a first hydrocarbon fuel with a first molar quantity of oxygen mixed with a second hydrocarbon fuel with a second molar quantity of oxygen, a second mixed fuel having a second mixture ratio of the first hydrocarbon fuel mixed with the second hydrocarbon fuel, the first ratio being different than the second ratio, and an equation that relates the fuel mixture ratio to the free oxygen content of the engine exhaust gas and to the engine airflow rate and engine fuel flow rate, the equation having a plurality of coefficients;
operating the engine with the first mixed fuel at a speed and torque and measuring the free oxygen of the exhaust gas, engine airflow rate, and engine fuel flow rate;
operating the engine with the second mixed fuel at a plurality of speeds and torques and measuring the free oxygen of the exhaust gas, engine airflow rate, and engine fuel flow rate at each speed and torque; and
using the measured data from said operating with the first mixed fuel and from said operating with the second mixed fuel to establish each of the coefficients.

40. The Method of claim 39 wherein the equation includes a first term interrelating the oxygen content, airflow rate, and fuel flow rate, a second term interrelating the airflow rate and the fuel flow rate, and a third term that is constant, and the first term is multiplied by a first coefficient, the second term is multiplied by a second coefficient, and the third coefficient is the constant, and the sum of the first term, second term, and third term corresponds to the mixture ratio.

41. The Method of claim 40 wherein the equation includes a fourth term comprising a fourth coefficient multiplying the oxygen content, and the fourth term is added to the first term, second term, and third term.

42. The method of claim 20 wherein the fuel mixture ratio can be expressed as the volumetric fuel blend fraction, and the general relationship is of the type:
$${B}_{vol} \approx {C}_{1} ( \frac{{x}_{{O}_{2}}}{f} ) + {C}_{2} ( \frac{1}{f} ) + {C}_{3}$$
where:
Bᵥₒₗ corresponds to the volumetric biofuel blend fraction,
χ_{O2} corresponds to the exhaust O₂ mole fraction,
f corresponds to the mixture fraction, and
C₁, C₂, and C₃ are constant coefficients.

43. The method of claim 42 wherein said modifying includes using the first data and the second data to determine at least one of C1, C2, or C3.

44. The method of claim 20 wherein the fuel mixture ratio can be expressed as the volumetric fuel blend fraction, and the general relationship is of the type:
$${B}_{vol} \approx {C}_{1} {x}_{{O}_{2}} ( \frac{{\dot{m}}_{air}}{{\dot{m}}_{fuel}} ) + {C}_{1} {x}_{{O}_{2}} + {C}_{2} ( \frac{{\dot{m}}_{air}}{{\dot{m}}_{fuel}} ) + {C}_{4}$$
where:
C₁, C₂, and C₄ are constant coefficients,
Bᵥₒₗ corresponds to the volumetric fuel blend fraction,
mdot(air) corresponds to the mass flowrate of air into the engine,
mdot(fuel) corresponds to the mass flowrate of fuel into the engine, and
χ_{O2} is the exhaust O₂ mole fraction.

45. The method of claim 44 wherein said modifying includes using the first data and the second data to determine at least one of C₁, C₂, C₅, or C₄.

46. The method of claim 28 wherein the term is:
$${C}_{1} ( \frac{{x}_{{O}_{2}}}{f} )$$
where:
χ_{O2} corresponds to the exhaust O₂ mole fraction,
f corresponds to the mixture fraction, and
C₁ is a constant coefficient.

47. The method of claim 28 wherein the term is one of the following:
$${C}_{1} \frac{{X}_{o 2} mdot ( air )}{mdot ( fuel )} or {C}_{1} {X}_{O 2}$$
where:
C₁ is a constant coefficient,
mdot(air) corresponds to the mass flowrate of air into the engine,
mdot(fuel) corresponds to the mass flowrate of fuel into the engine, and
χ_{O2} corresponds to the exhaust O₂ mole fraction.

48. The method of claim 10 which further comprises measuring the humidity of the air entering the engine and wherein said interpreting the ratio of the first fuel to the second fuel includes the humidity of the entering air.
